(19) United States
(12) Patent Application Publication
(10) Pub. No.: US 2011/0257446 A1
(43) Pub. Date: Oct. 20, 2011

(54) PROCESS FOR PRODUCING HYDROCARBONS FROM MICROBIAL LIPIDS

(76) Inventors: Carolina BOTELLA-FRANCO, Chester (GB); Hans De Lang, Oterleek (NL); Lavanya Meesala, Amsterdam (NL); Argi Joachim Antonio Moppi, Amsterdam (NL); Colin John Schaverien, Amsterdam (NL)

(21) Appl. No.: 12/972,120

(22) Filed: Dec. 17, 2010

(30) Foreign Application Priority Data
Dec. 18, 2009 (EP) 09179842.1

(51) Int. Cl. C07C 9/00; C07C 1/207

(52) U.S. Cl. 585/16; 585/310

(57) ABSTRACT

A process for producing hydrocarbons from microbial lipids is provided by: contacting a feed comprising microbial lipids, wherein the microbial lipids comprise a triglyceride content in the range of from 40 wt % to 70 wt % and a free fatty acid content in the range of from 10 wt % to 30 wt %, with a hydrogenation catalyst and hydrogen at a temperature in the range of from 250 to 380° C. and a total pressure in the range of from 20 to 160 bar (absolute), to obtain an effluent comprising paraffinic hydrocarbons and water; optionally separating a liquid stream rich in paraffinic hydrocarbons from the effluent; contacting the paraffinic hydrocarbons in the liquid stream rich in paraffinic hydrocarbons or the effluent comprising paraffinic hydrocarbons by contacting hydrogen and the liquid stream with hydroisomerisation catalyst at a temperature in the range of from 280 to 450° C. and a total pressure in the range of from 20 to 160 bar (absolute); and separating at least one product fraction from the product stream.

This application claims the benefit of European Application No. 09179842.1 filed Dec. 18, 2009 which is incorporated herein by reference.

### FIELD OF THE INVENTION

The present invention relates to a process for converting microbial lipids, in particular those derived from oleaginous microorganisms, to produce hydrocarbon fuel components.

### BACKGROUND OF THE INVENTION

With the diminishing supply of crude oil, the use of renewable energy sources is becoming increasingly important as a feedstock for production of hydrocarbon compounds. Plants, animal and microbial biomass are being used to produce liquid and gaseous hydrocarbon compounds. Microbial biomass includes components such as lipids, sugars, metabolites, etc. obtained from microorganisms like algae, yeast, fungi and bacteria. These microorganisms are being explored as feed for the production of hydrocarbon compounds primarily due to their easy cultivation and high purity yields.

US-A-20090047721 describes the use of fungi, algae and yeast for the production of fuels such as renewable diesel. Herein, the lipids present in the microorganisms are often extracted and further processed by transesterification for the production of fuels such as diesel or FAME. The most common disadvantage with the use of microorganisms for fuel production is the tedious procedure involved in the extraction and drying of lipids. Also, it is not possible to obtain a product with desirable properties due to the variability in the extracted lipid composition, and the obtained diesel components have a low cetane value, and cause issues with build up of components in engine oil of internal combustion engines. It is therefore evident that while the use of microorganisms for the production of fuels is well established, there is a need for a well developed, cost efficient process that yields fuels of good quality.

### SUMMARY OF THE INVENTION

A process for producing hydrocarbons from microbial lipids is provided, comprising the steps of:

(a) contacting a feed comprising microbial lipids, wherein the microbial lipids comprise a triglyceride content in the range of from 40 wt % to 70 wt % and a free fatty acid content in the range of from 10 wt % to 30 wt %, with a hydrogenation catalyst and hydrogen at a temperature in the range of from 250 to 380° C. and a total pressure in the range of from 20 to 160 bar (absolute), to obtain an effluent comprising paraffinic hydrocarbons and water;

(b) contacting at least a portion of the paraffinic hydrocarbons in the effluent comprising paraffinic hydrocarbons by contacting the liquid stream with hydrogen in the presence of a hydroisomerisation catalyst at a temperature in the range of from 280 to 450° C. and a total pressure in the range of from 20 to 160 bar (absolute); and

(c) separating at least one product fraction from the product stream obtained in step (b).

In a further embodiment a process to produce diesel fuel and/or kerosene fuel components is provided where the above process is used to obtain diesel fuel and/or kerosene fuel components in step (d).

### DETAILED DESCRIPTION

It has now been found that microbial lipids obtained from oleaginous microorganisms, preferably yeasts, can directly be converted into paraffinic kerosene and diesel components with excellent cold flow properties by applying a hydro-deoxygenation/hydro-isomerisation process. Such a process allows one to convert the microbial lipids to hydrocarbons without needing a conversion to C14-C18 alkyl esters as described in US2009/0047721.

In one embodiment, a process for producing hydrocarbons from microbial lipids is provided, comprising the steps of:

(a) contacting a feed comprising microbial lipids, wherein the microbial lipids comprise a triglyceride content in the range of from 40 wt % to 70 wt % and a free fatty acid content in the range of from 10 wt % to 30 wt %, with a hydrogenation catalyst and hydrogen at a temperature in the range of from 250 to 380° C. and a total pressure in the range of from 20 to 160 bar (absolute), to obtain an effluent comprising paraffinic hydrocarbons and water;

(b) optionally separating a liquid stream rich in paraffinic hydrocarbons from the effluent obtained in step (a);

(c) contacting the paraffinic hydrocarbons in the liquid stream rich in paraffinic hydrocarbons or the effluent comprising paraffinic hydrocarbons by contacting hydrogen and the liquid stream with hydroisomerisation catalyst at a temperature in the range of from 280 to 450° C. and a total pressure in the range of from 20 to 160 bar (absolute); and

(d) separating at least one product fraction from the product stream obtained in step (c).

Microbial oils, produced by oleaginous microorganisms including bacteria, algae and yeasts, have become a promising potential source for biodiesel production. Oleaginous microorganisms are defined as being able to accumulate lipids over 20 wt % of their dry biomass. A proportion of those lipids are in the form of triacylglycerols (TAGs), containing fatty acids that are comparable to those found in conventional vegetable oils. Yeasts are eukaryotic micro-organisms classified in the kingdom Fungi. Oleaginous yeasts or oil-producing yeasts produce and store lipids similar to vegetable oils and fats.

Microbial lipids as referred to in the present invention are a group of naturally occurring compounds that are usually hydrophobic and contain long-chain aliphatic hydrocarbons and their derivatives such as fatty acids, alcohols, amines, amino alcohols and aldehydes. These lipids include monoglycerides, diglycerides and triglycerides, which are esters of glycerol and fatty acids, and phospholipids, which are esters of glycerol and phosphate group-substituted fatty acids. The fatty acid moiety in the lipids used in the present invention ranges from 4 carbon atoms to 30 carbon atoms, and includes saturated fatty acids containing one, two or three double bonds. Preferably, the fatty acid moiety includes 8 carbon atoms to 25 carbon atoms, more preferably 12 carbon atoms to 20 carbon atoms. The lipids may contain variable amounts of free fatty acids and/or esters, both of which may also be converted into hydrocarbons during the process of this invention. The lipids typically also include carotenoids, hydrocarbons, phosphatides, simple fatty acids and their esters, terpenes, sterols,fatty alcohols, tocopherols, polyisoprene, carbohydraes and proteins, glycolipids and phospholidips.

The most preferred lipids as feedstock for the subject process are those obtained from the culturing of oleaginous yeasts such as Cryptococcus curvatus, Cryptococcus terricolus, Candida sp., Lipomyces starkeyi, Lipomyces lipofer, Endomycopsis vernalis, Rhodotorula glutinis, Rhodotorula gracilis, Rhodosporidium toruloides and Yarrowia lipolytica in suitable conditions of pH and temperature and in the presence of a suitable culture medium.

A particularly interesting oleaginous yeast, Rhodosporidium toruloides, was found to be one of the most promising microorganisms for the conversion of sugars to lipid considering variables such as lipid content, carbon efficiency, volumetric productivity and product yield. In particular R. toruloides 444 accumulated higher lipid content than other species of the strain tested, and to the high saturated content of the fatty acids in the lipids.

The subject process preferably also comprises the steps of (i) cultivating the oleaginous microbe at suitable conditions and with a suitable medium to produce microbial lipids, and (ii) extracting the microbial lipids from the microbe, prior to subjecting the lipids to step (a).

In step (i), the oleaginous microbe, more particularly the oleaginous yeast is preferably cultured in a fermentor at pH conditions in the range of from 5 to 6. The temperature maintained in the fermentor is preferably in the range of from 20 to 40° C., more preferably in a range of from 25 to 35° C. . An aeration of preferably about 1 vvm is preferably maintained in the fermenter. The culture medium used preferably comprises a basal medium, comprising a carbon source such as glucose, sucrose, xylose or any other suitable carbon source in a concentration of from 50 to 70 g/L, more preferably 60 g/L, peptone in a range of from 15 to 25, more preferably 19 to 21 g/L) and more preferably, yeast extract in a range of from 15 to 25, more preferably 19 to 21 g/L). The fermentor is preferably inoculated with a seed inoculum which has grown in two stage culture preferably in a YMY medium with a glucose source. The fermentation may preferably be run in fed-batch mode preferably using a concentrated carbon source solution, which is preferably fed when the initial carbon source is consumed. Several; different feeds may be added during the fermentation to increase carbon source concentration up to 100 g/L.

Suitable fermentation media includes Brazilian sugar cane juice, preferably diluted for the basal media up to 60 g/L and concentrated up to 800 g/L as a feeding solution.

Preferably, a feed-batch strategy is used, since it was found that yeast was able to accumulated between 40-70% depending on the feedstock used obtaining a yield of 26 g lipid per g of C source. The lipids thus obtained from the aforementioned fermentation process are preferably isolated from the microbial population for further processing. The lipids obtained from the oleaginous microbial species will be referred to as lipids for the purpose of the present invention. The microbial lipids preferably have a triglyceride content in the range of from 40 wt % to 70 wt % and a free fatty acid content in the range of from 10 wt % to 30 wt %.

The microbial lipids are then suitably extracted from the microbial cells. This may be done by any method that is suitable. Such methods are well known in the art, see for instance U.S. Pat. No. 6,727,373, US-A-200125114 and CA-A-2579516.

In accordance with the present invention, the thus obtained microbial lipids are subjected to a hydrogenation in the presence of a suitable hydrogenation catalyst, and optionally a hydrocarbon feed in a suitable hydrotreatment reactor.

In the process according to the invention, hydrogen and the feedstock comprising lipids are first contacted with a hydrogenation catalyst under hydro-deoxygenation conditions in step (a). In this hydrodeoxygenation step (a), triglycerides, diglycerides, monoglycerides and/or free fatty acids in the feedstock are converted into hydrocarbons, water and carbon oxides. The extent to which decarboxylation occurs depends on the hydrogenation catalyst used and the process conditions applied.

The hydro-deoxygenation conditions comprise a temperature in the range of from 250 to 380° C. and a pressure in the range of from 20 to 160 bar (absolute). Preferably, the hydro-deoxygenation temperature in step (a) is in the range of from 280 to 340° C. Reference herein to the hydro-deoxygenation temperature is to the maximum temperature that is occurring in hydro-deoxygenation step (a).

Since the hydro-deoxygenation reaction is a strongly exothermic reaction, the temperature in the upper part of the catalyst bed will typically be higher than the temperature in the upper lower part of the catalyst bed.

An effluent comprising paraffinic hydrocarbons and water is obtained in step (a). The effluent further comprises carbon oxides, unconverted hydrogen, and, if the feedstock comprises sulphur and/or nitrogen-containing compounds also hydrogen sulphide and/or ammonia.

A suitable catalyst will be present in the hydrotreatment unit.

The feed to the hydrodeoxygenation reactor may include hydrocarbons derived from mineral crude oil or refinery streams. Preferably, the hydrocarbon feed used has a boiling point of at least 220° C., as measured by Gas Chromatograph Distillation (GCD) according to ASTM D-2887. More preferably, the boiling point range from 220° C. to 650° C., yet more preferably from 300° C. to 600° C. The hydrocarbon feed for the purpose of the present invention includes high boiling, non-residual oils such as straight run (atmospheric) gas oils, flashed distillate, coker gas oils, or atmospheric residue (‘long residue’) and vacuum residue (‘short residue’). More preferably, mainly paraffinic and/or naphthtenic compounds such as dodecane or hydrotreated gas oil can be used as hydrocarbon feed, since they are reasonably inert and only exhibit minor cracking and/or hydrogenation at the conditions of the hydrodeoxygenation, but permit to dilute the heat of the reaction to avoid hotspots. Alternatively, part of the liquid product stream obtained in optional step (b) can be recycled as diluents to step (a).

In operation, the hydrotreatment unit includes an arrangement of heaters, heat exchangers, reactors, compressors and boilers that are set at different temperatures and pressures. Preferably, the yeast lipids and the hydrocarbon feed can be blended and introduced into the hydrotreatment unit as the feedstock through a charge pump. The hydrocarbon feed may also be introduced in the hydrotreatment unit at different stages. When operating in a co-feed mode, the hydrocarbon feed to the yeast lipids ratio ranges from 1:100 to 100:1, preferably 20:1 to 1:20, more preferably from 5:1 to 1:5.

The feedstock, whether neat lipids or blended with a hydrocarbon feed, is preferably mixed with hydrogen gas which can be directed from a catalytic reforming unit or a hydrogen plant. The feedstock and the hydrogen mixture are preferably heated in succession through heat exchange with reactor effluent in a heat exchanger unit and a fired heater.

The number of reactor volumes of feed that can be treated in a unit time is indicative by space velocity which is preferably 0.5 kg/l/h to 2 kg/l/h and rate at which the hydrogen gas flows also referred to as gas rate is kept preferably at 800 to 2000 Nl/kg/h.

Catalysts suitable for use in step (a) of the process according to the invention are well known in the art. The catalyst usually comprise two parts, a catalyst support and active elements. The support comprises solid substances with high porosity and able to withstand the temperature, pressure and the environment encountered in the hydrotreatment unit. For example, alumina in the form of balls or extrudates can be used as a support for the active elements in the catalyst. The active elements used are preferably cobalt, more preferably, nickel, molybdenum, tungsten and its combinations thereof.

Preferably, the hydrogenation catalyst comprises sulphided hydrogenation compounds, typically sulphided nickel or cobalt in combination with sulphided molybdenum or tungsten. In case of such sulphided hydrogenation catalyst, a sulphur source will typically be supplied to the hydrogenation catalyst in order to keep the catalyst in sulphided form during hydrodeoxygenation step (a). As a consequence, the effluent of step (a) then comprises hydrogen sulphide.

The hydrogenation catalyst of step (a) may be any hydrogenation catalyst known in the art that is suitable for hydro-deoxygenation, typically a catalyst comprising metals of Group VIII and/or Group VIB of the Periodic Table of Elements or compounds thereof. Examples of such catalysts are catalysts comprising Pd, Pt, reduced Ni, or sulphided CoMo, NiMo or NiW as hydrogenation components on a carrier. The carrier typically comprises a refractory oxide, preferably alumina, amorphous silica-alumina, titania or silica. The carrier may comprise a zeolitic compound.

If a catalyst comprising sulphided CoMo, NiMo or NiW is used, the catalyst may be sulphided in-situ or ex-situ. In-situ sulphiding may be achieved by supplying a sulphur source, usually hydrogen sulphide or a hydrogen sulphide precursor, i.e. a compound that easily decomposes into hydrogen sulphide such as for example dimethyl disulphide, di-tert-nonyl polysulphide or di-tert-butyl polysulphide, to the catalyst of step (a) during operation of the process. The sulphur source may be supplied with the feedstock, the hydrogen or separately. An alternative suitable sulphur source is a sulphur-comprising hydrocarbon stream boiling in the diesel or kerosene boiling range that is be co-fed with the feedstock. Preferably, an amount of in the range of from 100 to 5,000 ppmv hydrogen sulphide, more preferably of from 500 to 2,000 ppmv, or an equivalent amount of a hydrogen sulphide precursor, based on the volume of hydrogen supplied, is supplied to step (a).

In optional separation step (b), a liquid stream rich in paraffinic hydrocarbons is separated from the effluent obtained in step (a). Preferably, separation step (b) is carried out at a high pressure, i.e. a pressure in the range of from 0.5 to 10 bar lower, preferably of from 1 to 5 bar lower, than the pressure at the outlet of the reactor vessel in which step (a) is carried out.

In the optional separation step may be carried out in a low temperature, high pressure separator to separate a gaseous stream depleted in water, a liquid water-rich stream and the liquid stream rich in paraffinic hydrocarbons from the effluent obtained in step (a). Low temperature, high pressure separators are known in the art. In the low temperature, high pressure separator, the effluent of step (a) is first cooled, preferably to a temperature in the range of from 10 to 150° C., and the cooled effluent is then, in a separation vessel, separated into a gaseous phase depleted in water and a liquid phase. Due to a difference in density, the liquid phase separates into a water-rich liquid phase and hydrocarbon-rich liquid phase. The pressure in the separation vessel is preferably in the range of from 0.5 to 10 bar lower, more preferably in the range of from 1 to 5 bar lower, than the total pressure at the outlet of the reactor vessel wherein step (a) is carried out.

The gaseous stream depleted in water obtained in the low temperature, high pressure separator of step (b) may be recycled, optionally after removal of impurities like hydrogen sulphide, ammonia, carbon oxides, light hydrocarbons or steam, to step (a) and/or step (b) to provide part of the hydrogen needed in step (a) and/or step (b). Alternatively, optional step (b) may be carried out in a high temperature, high pressure separator to separate a gaseous stream rich in water and the liquid stream rich in paraffinic hydrocarbons from the effluent obtained in step (a). High temperature, high pressure separators are known in the art. It will be appreciated that the temperature in the high temperature, high pressure separator is chosen such that there is sufficient separation between water and paraffinic hydrocarbons whilst the temperature is as little as possible below the inlet temperature of hydroisomerisation step (c). Typically, the temperature in the high temperature, high pressure separator is in the range of from 160 to 350° C., usually of from 180 to 320° C. The gaseous stream rich in water will contain the major part of the water that was present in the effluent of step (a). If the gaseous stream is to be recycled to step (a) and/or step (b), it is therefore preferred that water is removed from it prior to recycling. Water removal from the gaseous stream rich in water is suitably done in a low temperature, high pressure separator. Thus, a gaseous stream depleted in water is obtained that may be recycled, optionally after removal of impurities like hydrogen sulphide, ammonia, carbon oxides, light hydrocarbons or steam, to step (a) and/or step (b) to provide part of the hydrogen needed in step (a) and/or step (b).

If no separation step is applied then care has to be taken to not allow the water to condense as this may negatively impact the catalyst particle strength, and lead to metal leaching in step (b).

The catalyst of step (b) may comprise a zeolitic compound. Any acidic zeolitic compound having hydro-isomerising activity may suitably be used. Such zeolitic compounds are known in the art. Examples of such zeolitic compounds include, but are not limited to, zeolite Y, zeolite beta, ZSM-5, ZSM-12, ZSM-22, ZSM-23, ZSM-48, SAPO-11, SAPO-41, and ferrierite.

The hydrocarbon product stream obtained from the hydrogenation step comprises mainly normal paraffins in the range of C₈ to C₂₀ and is further subjected to a catalytic isomerisation step. Catalytic isomerisation as referred to in the present invention is the rearrangement of atoms within a molecule in the presence of a catalyst. It is typically used for upgrading of hydrocarbons such that they can effectively be used as fuels. The catalytic isomerisation of longer chain hydrocarbons is used to increase the cold flow properties.

The liquid stream rich in paraffinic hydrocarbons obtained in optional separation step, or the total effluent of step (a)is hydroisomerised in step (b). If the separation is applied, preferably, the liquid stream comprises less than 30 wt %, more preferably less than 10 wt %, even more preferably less than 5 wt %, of the water comprised in the effluent of step (a). The liquid stream may further comprise impurities like propane, dissolved hydrogen sulphide, and carbon oxides. It will be appreciated that the lower the temperature in separation step the higher the amount of low-molecular weight compounds dissolved in the liquid stream rich in paraffinic hydrocarbons.

In step (b), the catalytic isomerisation is typically conducted in the presence of an isomerisation catalyst which maybe a molecular sieve-based catalyst which exhibits selective and substantial isomerisation activity under the operating conditions of the isomerisation zone. For the purpose of the present invention, the isomerisation catalyst used is preferably a nickel/tungsten (Ni/W) based catalyst, while under sulphur free conditions a platinum based dewaxing catalyst may be used. The catalyst composition comprises at least a hydrogenation component, a binder and zeolite crystallites, wherein the zeolite has pores consisting of 12 oxygen atoms and has a constrain index (CI) larger than 1. The method by which the CI value according to this invention is determined is described in U.S. Pat. No. 4,016,218. It should be noted that Constraint Index seems to vary somewhat with severity of operations (conversion) and the presence or absence of binders. Likewise, other variables, such as crystal size of the zeolite, the presence of occluded contaminants, etc., may affect the Constraint Index. Therefore, it will be appreciated that it may be possible to so select test conditions, e.g. temperature, as to establish more than one value for the Constraint Index of a particular zeolite. This explains the range of Constraint Indices for some zeolites, such as ZSM-5, ZSM-11 and Beta. For the purposes of the present invention, a zeolite is considered to have a Constraint Index of larger than 1 if when tested at at least one temperature within the range of 550° F. (290° C.) to 950° F. (570° C.), it manifests a Constraint Index within the here specified ranges. The CI value is greater than 1, preferably greater than 1.5. The maximum value for the CI will be suitably smaller than 12 and preferably smaller than 7. The zeolite used in the present invention is not a typical large pore zeolites such as zeolite beta (BEA type) or mordenite (MOR type) because typically such large pore zeolites have a CI value of less than 1. The three letter code describing the zeolite is according to the Structure Type Codes as defined by the IZA Structure Commission and described in detail in Zeolites 17:1-230, 1996 pages 5-12. Nor is the zeolite a typical medium pore zeolite because medium pore zeolites typically have pores consisting of 10 oxygen atoms as the largest pore opening. Such medium pore zeolites typically have a CI value larger than 1, for example ZSM-23 (MTT Type) having a CI value of 9.1.

More preferably the zeolite has 12 oxygen-ring defined pores, wherein the largest pore axis of these pores is between 5 and 7 Å. This axis length should be determined by X-ray diffraction. Typical values for such axis are described for different zeolites in Zeolites 17:1-230, 1996 page 9.

Examples of zeolites, which can be used in the present invention having the above properties, are zeolites of the OFF Type and MTW type zeolites. Both these 12-oxygen ring zeolites have CI value's of above 1 and more preferably above 1.5. Examples of OFF type zeolites are Linde T, LZ-217 and TMA-O. Reference is also made to U.S. Pat. No. 4,503,023 describing an OFF Type zeolite. More preferably MTW type zeolites are used. This class of zeolites includes ZSM-12 as described in U.S. Pat. No. 3,832,449, CZH-5 as described in GB-A-2079735, Gallosilicate MTW as described in Y. X. Zhi, A. Tuel, Y. Bentaarit and C. Naccache, Zeolites 12, 138 (1992), Nu-13(5) as described in EP-A-59059, Theta-3 as described in EP-A-162719, TPZ-12 as described in U.S. Pat. No. 4,557,919 and VS-12 as described in K. M. Reddy, I. Moudrakovski and A. Sayari, J. Chem. Soc., Chem. Commun. 1994, 1491 (1994). The average crystal size of the zeolite is preferably smaller than 0.5 μm and more preferably smaller than 0.1 μm as determined by the well-known X-ray diffraction (XRD) line broadening technique using the high intensity peak at about 20.9 2-theta in the XRD diffraction pattern.

The binder in the catalyst may be any binder usually used for such an application. A possible binder includes alumina or alumina containing binders. Applicants have found that low acidity refractory oxide binder material that is essentially free of alumina provides more improved catalyst. Examples are low acidity refractory oxides such as silica, zirconia, titanium dioxide, germanium dioxide, boria and mixtures of two or more of these. The most preferred binder is silica. The weight ratio of the molecular sieve and the binder can be anywhere between 5:95 and 95:5. Lower zeolite content, suitable between 5 and 35 wt %, may in some cases be advantageous for achieving an even higher selectivity.

The silica to alumina molar ratio of the zeolite prior to dealumination is preferably larger than 50 and more preferably between 70 and 250 and most preferably between 70 and 150. Preferably the zeolite has been subjected to a dealumination treatment. The dealumination of the zeolite results in a reduction of the number of alumina moieties present in the zeolite and hence in a reduction of the mole percentage of alumina. The expression “alumina moiety” as used in this connection refers to an Al₂O₃-unit which is part of the framework of the aluminosilicate zeolite, i.e. which has been incorporated via covalent bindings with other oxide moieties, such as silica (SiO₂), in the framework of the zeolite. The mole percentage of alumina present in the aluminosilicate zeolite is defined as the percentage of moles Al₂O₃ relative to the total number of moles of oxides constituting the aluminosilicate zeolite (prior to dealumination) or modified molecular sieve (after dealumination). Preferably dealumination is performed such that the reduction in alumina moieties in the framework is between 0.1 and 20%.

Dealumination may be performed by means of steaming. Preferably the surface of the zeolite crystallites are selectively dealuminated. A selective surface dealumination results in a reduction of the number of surface acid sites of the zeolite crystallites, whilst not affecting the internal structure of the zeolite crystallites. When applying a surface dealumination the reduction of alumina moieties in the framework will be lower and preferably between 0.1 and 10%. Dealumination using steam results is a typical non-selective dealumination technique.

Dealumination can be attained by methods known in the art. Particularly useful methods are those, wherein the dealumination selectively occurs, or anyhow is claimed to occur selectively, at the surface of the crystallites of the molecular sieve. Examples of dealumination processes are described in WO-A-9641849. U.S. Pat. No. 5,015,361 describes a method wherein the zeolites are contacted with sterically hindered amine compound.

Preferably dealumination is performed by a process in which the zeolite is contacted with an aqueous solution of a fluorosilicate salt wherein the fluorosilicate salt is represented by the formula:

(A)_{2/b}SiF₆

wherein ‘A’ is a metallic or non-metallic cation other than H⁺ having the valence ‘b’. Examples of cations ‘b’ are alkylammonium, NH4⁺, Mg⁺⁺, Li⁺, Na⁺, K⁺, Ba⁺⁺, Cd⁺⁺, Cu⁺, Ca⁺⁺, Cs⁺, Fe⁺⁺, Co⁺⁺, Pb⁺⁺, Mn⁺⁺, Rb⁺, Ag⁺, Sr⁺⁺, Tl⁺, and Zn⁺⁺. Preferably ‘A’ is the ammonium cation. The zeolite material may be contacted with the fluorosilicate salt at a pH of suitably between 3 and 7. Such a dealumination process is for example described in U.S. Pat. No. 5,157,191. The dealumination treatment is also referred to as the AHS-treatment.

The catalyst composition is preferably prepared by first extruding the zeolite with the low acidity binder and subsequently subjecting the extrudate to a dealumination treatment, preferably the AHS treatment as described above. It has been found that an increased mechanical strength of the catalyst extrudate is obtained when prepared according to this sequence of steps. It is believed that by maintaining the acidity of the catalyst at a low level conversion to products boiling outside the lube boiling range is reduced. Applicants found that the catalyst should have an alpha value below 50 prior to metals addition, preferably below 30, and more preferably below 10. The alpha value is an approximate indication of the catalytic cracking activity of the catalyst compared to a standard catalyst. The alpha test gives the relative rate constant (rate of normal hexane conversion per volume of catalyst per unit time) of the test catalyst relative to the standard catalyst which is taken as an alpha of 1 (Rate Constant=0.016 sec −1). The alpha test is described in U.S. Pat. No. 3,354,078 and in J. Catalysis, 4, 527 (1965); 6, 278 (1966); and 61, 395 (1980), to which reference is made for a description of the test. The experimental conditions of the test used to determine the alpha values referred to in this specification include a constant temperature of 538° C. and a variable flow rate as described in detail in J. Catalysis, 61, 395 (1980).

The hydrogenation component in step (c) suitably comprises at least one Group VIB metal component and/or at least one Group VIII metal component. Group VIB metal components include tungsten, molybdenum and/or chromium as sulphide, oxide and/or in elemental form. If present, a Group VIB metal component is suitably present in an amount of from 1 to 35% by weight, more suitably from 5 to 30% by weight, calculated as element and based on total weight of support, i.e. modified molecular sieve plus binder. Group VIII metal components include those components based on both noble and non-noble metals.

Particularly suitable Group VIII metal components, accordingly, are palladium, platinum, nickel and/or cobalt in sulphidic, oxidic and/or elemental form. Nickel and/or cobalt, if present at all, may be present in an amount in the range of from 1 to 25% by weight, preferably 2 to 15% by weight, calculated as element and based on total weight of support. The total amount platinum or palladium will suitably not exceed 10% by weight calculated as element and based on total weight of support, and preferably is in the range of from 0.1 to 5.0% by weight, more preferably from 0.2 to 3.0% by weight. If both platinum and palladium are present, the weight ratio of platinum to palladium may vary within wide limits, but suitably is in the range of from 0.05 to 10, more suitably 0.1 to 5. Catalysts comprising palladium and/or platinum as the hydrogenation component are preferred. Most preferred is when platinum is used as the sole hydrogenation component. The hydrogenation component is suitably added to the catalyst extrudate comprising the dealuminated aluminosilicate zeolite crystallites by known techniques. Step (c) is typically performed at a temperature in the range of from 280 to 450° C. and a total pressure in the range of from 20 to 160 bar (absolute). Preferably, the hydro-isomerisation temperature is in the range of from 300 to 400° C., more preferably of from 330 to 380° C. The total pressure in each of steps (a) and (c) is preferably in the range of from 40 to 120 bar (absolute), more preferable of from 50 to 80 bar (absolute).

The weight hourly space velocities (WHSV) in the range of from 0.1 to 10 kg of oil per litre of catalyst per hour (kg/l/hr), preferably from 0.2 to 5 kg/l/hr, more preferably from 0.5 to 3 kg/l/hr and hydrogen to oil ratios in the range of from 100 to 2,000 litres of hydrogen per litre of oil. It is appreciated that hydrogenation (step a) and catalytic isomerisation (step c) can be carried out simultaneously in a reactor with a stacked bed single stage configuration that enables the step (a) to take place on one bed with the hydrogenation catalyst stacked on the bed. The product stream obtained in step (a) is then fed to another bed of the reactor stacked with isomerisation catalyst where it is subjected to step (c).

The product stream, obtained from step (c) is further subjected to a separation process (d) to primarily obtain renewable diesel and/or kerosene fractions along with other hydrocarbons that may be processed further. The commercial products obtained from these hydrocarbons are also within the scope of the invention. It may be understood that processing of the aforementioned hydrocarbon products is well known in the art and is in no way limiting to the scope of the invention. While some of the methods have been described herein, several other processes may be used to convert the hydrocarbon fractions into commercially usable products. These processes may include desulfurization, cracking into more valuable lighter products, blending with other fuels for commercial use, and other similar uses that have been disclosed in the art.

### EXAMPLE 1

The oleaginous yeast R. toruloides 444 was cultured in a fermentor at pH conditions in the range of from 5 to 6, at a temperature maintained of 30° C. An aeration of about 1 vvm was maintained in the fermentor. The culture medium used comprised a basal media comprising a carbon source based on glucose in a concentration of 60 g/L, peptone (20.3 g/L) and yeast extract (20.3 g/L). The fermentor was inoculated with a seed innoculum which has grown in two stage culture preferably in a YMY medium with a glucose source. The fermentation was operated in fed-batch mode using a concentrated carbon source solution was fed when the initial carbon source was consumed. Three different feeds were added during the fermentation to increase carbon source concentration up to 100 g/L. After the first feeding when the concentration of nitrogen was consumed, the yeast started accumulate lipids. The yeast was able to accumulated between 40-70% depending on the feedstock used obtaining a yield of 26 g lipid per g of C source.

The yeast lipids were extracted after cell disruption and centrifugation to remove the lipids, followed by a solvent extraction from the aqueous medium.

The yeast lipids comprised of 54 wt % triacylglycerides (TAGs), 3.9 wt % diglycerides, 0.2 wt % monoglycerides and 19.5 wt % of free fatty acids. The total metal content of the yeast lipids was 710 ppm, in particular phosphorus 300 ppm, silicon 250 ppm, sodium 90 ppm and calcium 41 ppm.

### EXAMPLE 2

The yeast lipids were diluted (18 wt %) in dodecane and fed to the supplied to the top bed of a fixed bed microflow unit at 300° C., 60 bar and WHSV (Weight by hour space velocity) of 1.0 g oil per mL catalyst of the top bed per hour. From the liquid sample analysis, the conversion of TAGs was 100%.

A gas stream comprising 2.5 vol % hydrogen sulphide and 97.5 vol % hydrogen was supplied to the top bed at a gas-to-oil ratio of 2000 NL/kg. The total pressure was 60 bar (a) in both beds.

The conversion based on amount of residual oxygen in liquid samples was 98.6-99.8%. As well as the dodecane used as diluents, the product contained 55 wt. % C₁₇, C₁₈ paraffins and 30 wt % Cl₅, C₁₆ paraffins. The results are illustrated in table 1.

The reactor contained 10 mL of a conventional hydrotreating catalyst comprising 3.5 wt % NiO and 15 wt % MoO₃ on a support of alumina, was placed above 10 mL of a catalyst comprising 5 wt % NiO and 21 wt % W₂O₃ on amorphous silica-alumina. Both catalysts were 1:1 diluted with 0.1 mm diameter silicon carbide spheres. The temperature of each bed was independently controlled by means of an oven. The temperature of the top bed was set at 300° C.; the temperature of the bottom bed at 350-370° C. The degree of isomerisation of the liquid effluent of the reactor was determined by gas chromatography.

TABLE 1
Wt % of iso and normal paraffins products

| Carbon chain | Percentage of products (Wt %) | |
|---|---|---|
| length | Non Branched | Branched |
| C4 | 0.006 | 0.123 |
| C5 | 0.004 | 0.004 |
| C6 | 0.005 | 0.007 |
| C7 | 0.018 | 0.033 |
| C8 | 0.024 | 0.084 |
| C9 | 0.006 | 0.047 |
| C10 | 0.011 | 0.016 |
| C11 | 0.010 | 0.006 |
| C12* | 85.050 | 0.405 |
| C13 | 0.118 | 0.016 |
| C14 | 0.125 | 0.011 |
| C15 | 1.933 | 0.011 |
| C16 | 1.898 | 0.017 |
| C17 | 4.971 | 0.073 |
| C18 | 4.507 | 0.086 |
| C19 | 0.065 | 0.029 |
| C20 | 0.043 | 0.008 |
| C21 | 0.037 | 0.001 |
| C22 | 0.033 | 0.003 |
| C23 | 0.050 | 0.006 |
| C24 | 0.044 | 0.008 |
| Total > C24 | 0.048 | |
| Total <= C24 | 98.957 | 0.995 |

*including 82% dodecane in feed.

The paraffinic products obtained after the hydrogenation stage were not considered suitable as fuel components for diesel and/or kerosene fuels since the cold flow properties of the mainly n-paraffinic feed are not suitable for diesel or kerosene use. However, the diesel and kerosene fractions of the liquid stream obtained after the two-stage process showed strongly improved cold flow properties.

We claim:

1. A process for producing hydrocarbons from microbial lipids, comprising the steps of:
(a) contacting a feed comprising microbial lipids, wherein the microbial lipids comprise a triglyceride content in the range of from 40 wt % to 70 wt % and a free fatty acid content in the range of from 10 wt % to 30 wt %, with a hydrogenation catalyst and hydrogen at a temperature in the range of from 250 to 380° C. and a total pressure in the range of from 20 to 160 bar (absolute), to obtain an effluent comprising paraffinic hydrocarbons and water;
(b) contacting at least a portion of the paraffinic hydrocarbons in the effluent comprising paraffinic hydrocarbons by contacting the liquid stream with hydrogen in the presence of a hydroisomerisation catalyst at a temperature in the range of from 280 to 450° C. and a total pressure in the range of from 20 to 160 bar (absolute); and
(c) separating at least one product fraction from the product stream obtained in step (b).

2. The process of claim 1 further comprising separating a liquid stream rich in paraffinic hydrocarbons from the effluent obtained in step (a); and contacting the paraffinic hydrocarbons in the liquid stream with hydrogen and in the presence of hydroisomerisation catalyst at a temperature in the range of from 280 to 450° C. and a total pressure in the range of from 20 to 160 bar (absolute)

3. The process of claim 1 further comprising isolating the microbial lipids from a microbial population.

4. The process of claim 1 wherein the feed comprises yeast-lipids in the range of from 2 wt % to 30wt %, preferably in the range of from 5 wt % to 20 wt %.

5. The process of claim 1 wherein the feed further comprises a hydrocarbon co-feed derived from mineral crude oil.

6. The process of claim 5 wherein the hydrocarbon co-feed is a hydrotreated gas oil fraction.

7. The process of claim 1 wherein the hydrotreatment catalyst of step (a) and/or step (b) comprises sulfided Ni and sulfided W or Mo as hydrogenation components on a carrier comprising amorphous silica-alumina and/or a zeolitic compound.

8. The process of claim 1 where the microbial lipids are obtained from a yeast selected from the group consisting of Cryptococcus curvatus, Cryptococcus terricolus, Candida sp., Lipomyces starkeyi, Lipomyces lipofer, Endomycopsis vernalis, Rhodotorula glutinis, Rhodotorula gracilis, Rhodosporidium toruloides and Yarrowia lipolytica.

9. The process of claim 7 where the microbial lipids are obtained from Rhodosporidium toruloides.

10. A kerosene or diesel fuel fraction produced by teh process of claim 1.

11. A kerosene or diesel fuel fraction produced by teh process of claim 2.

12. A kerosene or diesel fuel fraction produced by teh process of claim 8.
